(19) United States
(12) Patent Application Publication
(10) Pub. No.: US 2016/0058305 A1
(43) Pub. Date: Mar. 3, 2016

(54) DEVICE AND METHOD FOR ANALYZING REPERFUSION INJURY

(71) Applicant: University of Washington, Seattle, WA (US)

(72) Inventors: Graham Nichol, Mercer Island, WA (US); Adeyinka Adedipe, Seattle, WA (US); Pierre D. Mourad, Seattle, WA (US)

(21) Appl. No.: 14/836,022

(22) Filed: Aug. 26, 2015

Related U.S. Application Data
(60) Provisional application No. 62/042,035, filed on Aug. 26, 2014.

(51) Int. Cl. A61B 5/02; A61B 8/06; A61B 5/0225; A61B 5/00; A61B 8/00; A61B 5/026; A61B 5/022

(52) U.S. Cl. CPC A61B 5/02007; A61B 5/026; A61B 8/06; A61B 5/02233; A61B 5/7282; A61B 8/4483; A61B 5/0225; A61B 5/742; A61B 5/7475

(57) ABSTRACT

A reperfusion injury detection device comprising a measuring probe with a plurality of sensors collecting a hemodynamic property of a blood vessel of a subject coupled to a pneumatic cuff for applying transient occlusion to the blood vessel being measured and a reperfusion injury analysis device made up from a data module that measures the collected hemodynamic property and determines metric of the measured property over time, and display the measured property on an output module.

### CROSS-REFERENCE TO RELATED APPLICATION

This application claims the benefit, under 35 U.S.C. §119(e), of U.S. provisional patent application No. 62/042,035 filed Aug. 26, 2014 entitled “Novel Reperfusion Injury Sensor” which is hereby incorporated by reference in its entirety.

### BACKGROUND OF THE INVENTION

Proper restoration of blood flow during or following acute local (e.g. myocardial infarction or heart attack) or global (e.g. cardiac arrest) or chronic ischemia (e.g. myocardial stunning or heart failure), hereafter referred to as cardiac distress events, is essential to ensure survival of the subject experiencing the event. Restoration of blood flow can, however, induce reperfusion injury in the subject, such as through the release of inflammatory molecules and deterioration of the endothelium (the layer of cells lining an interior surface of a blood vessel). Notably, subjects that die from cardiac distress events typically have higher concentrations of inflammatory molecules like plasma interleukin 6 (IL-6) and tumor necrosis factors (TNF) relative to subjects that survive. The introduction of these higher concentrations contribute to changes of the coagulation of the blood through blood vessels and poor capillary function, tissue ischemia, organ dysfunction and can lead to death. Prompt treatment of reperfusion injury improves survival rates and neurological outcomes of subjects suffering cardiac distress events. Prompt detection of reperfusion injury is therefore needed.

### SUMMARY OF THE INVENTION

A medical device for detecting reperfusion injury and methods for analyzing reperfusion injury in a subject following restoration of blood flow related to cardiac distress events are described. Embodiments of the present invention relate to technologies for determining the incidence and severity of reperfusion injury through the use of non-invasive sensors coupled to a device applying transient occlusion to a blood vessel of a subject that has experienced, or is experiencing, a cardiac distress event such as ischemia or cardiac arrest.

Detection of reperfusion injury is most commonly correlated by analysis of flow mediated dilation (FMD) in a blood vessel, by which a transient occlusion is applied to a blood vessel and a diameter of the blood vessel is compared to pre-occluded and occluded states. Reliability of FMD data or other proxy hemodynamic property is inconsistent, however, due to user variability in location of the transient occlusion, location of the measurement of FMD or other proxy hemodynamic property, and duration of the transient occlusion. Ultrasonic transducers can improve FMD analysis, but for ambulatory subjects there has yet to be a portable and non-invasive means for reducing the variability in FMD or other proxy hemodynamic property data. Indeed, the current state of the art for such analysis is stereotactic (minimally invasive) devices such as Unex Co.'s UNEXEF18G, which is not only not non-invasive but not mobile or portable. Other non-invasive ways to detect reperfusion injury include measuring end tidal carbon dioxide, tissue acidosis, or lactate levels of a subject's blood, but these are limited to bedside analyses settings. Still other currently available non-invasive reperfusion detection means can be provided at the ambulatory or initial points of care (such as arterial tonometry to measure reactive hyperemia (the increase in blood flow after ischemia)) but these do not detect the magnitude of reperfusion injury, only the presence, and therefore limit the scope of appropriate early treatment.

Devices that can be rapidly applied to a subject, and quickly and reliably communicate the presence and degree of reperfusion injury with consistent reliability can greatly improve post-cardiac distress survival rates and neurological outcomes by prompting early treatment appropriate for, and responsive to, the subject's condition.

One embodiment of the invention is an array of non-invasive ultrasonic transducers placed over an artery and coupled to a pneumatic cuff. The transducers can collect a hemodynamic property, such as blood flow velocity or blood vessel diameter. The pneumatic cuff can apply transient occlusion to the blood vessel, such that the transducers collect the hemodynamic property before, during, and after the occlusion. A reperfusion injury analysis device can calculate a metric of the hemodynamic property such as blood vessel dilation changes (FMD) or changes in blood flow velocity or the time required for the post-occluded hemodynamic property to return to the pre-occluded hemodynamic property, and communicate the metric as a proxy for the presence and degree of reperfusion injury. Such communication enables prognosis, assessment and treatment of reperfusion injury at the initial or ambulatory point of care rather than requiring a hospital or other bedside medical care setting.

Embodiments of the invention improve reliability of analyzing FMD or suitable hemodynamic property by imparting consistent placement of sensors over blood vessels of a subject through the use of reference indicia on the sensors, such as ultrasonic transducers, and reference indicia on the pneumatic cuff applying transient pressure as well. The sensors and the cuff are operably coupled and controlled by a reperfusion injury analysis device that dictates the amount and duration of pressure applied, and coordinates the timing of the measurement relative to the occlusion. In certain embodiments, an adhesive gel between the sensors and the subject both improves transmission of signals between the sensors and blood vessel of a subject, as well as fixes the sensor to the subject throughout measurement. These embodiments not only reduce the variability currently in the art of analyses of FMD or other hemodynamic property, but provide a noninvasive and portable means of doing so.

Other embodiments of the present invention are methods for detecting the degree of reperfusion injury present in a subject that has experienced, or is experiencing, a cardiac distress event. In these embodiments, a hemodynamic property of a blood vessel is measured. A transient occlusion is then applied and the post-occluded hemodynamic property is measured. Metrics of the pre-occluded and post-occluded hemodynamic property are calculated and compared, or the time required to return the post-occluded property to the pre-occluded property is calculated. The calculated metric, in certain embodiments, is communicated at the point of care to prompt immediate prognosis and treatment as appropriate.

These and other embodiments of the invention along with many of its advantages and features are described in more detail in conjunction with the text below and attached figures.

### BRIEF DESCRIPTION OF THE DRAWINGS

The foregoing aspects and many of the attendant advantages of this invention and its embodiments will become more readily apparent when the accompanying detailed description is taken in conjunction with the following figures, which are illustrative only and not drawn to scale.

FIG. 1 illustrates a reperfusion device with a measuring probe applied to a brachial artery, and a pneumatic cuff applied to the humerus region of a subject, both coupled to a reperfusion injury analysis device according to a particular embodiment;

FIGS. 2A-2C illustrate a measuring probe with an application face housing a plurality of sensors, a measuring probe with an application face comprising an offset apparatus housing a plurality of sensors, and a measuring probe with reference indicia to align the measuring probe with an anatomical or external reference to place the measuring probe over a target blood vessel of a subject according to particular embodiments;

FIG. 3 illustrates a pneumatic cuff for applying transient occlusion with reference indicia to align the cuff with an anatomical reference or target occlusion point of a subject according to a particular embodiment;

FIG. 4 illustrates a system diagram of a reperfusion injury analysis device comprising a data module for measuring a hemodynamic property collected by a measuring probe and calculating a metric for the hemodynamic property, an output module for communicating the calculated metric of the hemodynamic property, and a controller for coordinating the instigation and duration of transient occlusion relative to measuring a hemodynamic property according to a particular embodiment;

FIG. 5 illustrates an exemplary method of measuring a hemodynamic property related to a blood vessel of a subject before applying transient occlusion, applying transient occlusion to the blood vessel being measured, measuring a hemodynamic property of a blood vessel of a subject after applying the transient occlusion, and calculating a metric relative to the pre-occluded and post-occluded hemodynamic property according to a particular embodiment;

FIG. 6 illustrates an exemplary method of communicating a metric of a pre-occluded and post-occluded hemodynamic property of a blood vessel of a subject according to a particular embodiment.

### DETAILED DESCRIPTION OF THE INVENTION

Embodiments of the present invention relate to technologies to quickly and reliably communicate the presence and severity of reperfusion injury in a subject that has experienced, or is experiencing, a cardiac distress event which include but are not limited to acute local (e.g. myocardial infarction or heart attack) or global (e.g. cardiac arrest) or chronic ischemia (e.g. myocardial stunning or heart failure). Embodiments of the invention enable prognosis, assessment, and treatment of reperfusion injury at the ambulatory or initial point of care.

In one embodiment, a measuring probe is applied proximate to a blood vessel of a subject. In one embodiment, the blood vessel is the brachial artery of the subject. Brachial artery properties are highly correlated with coronary properties. In another embodiment, the blood vessel is the radial artery of the subject. One of skill in the art can envision other appropriate blood vessels for applying a measuring probe to determine hemodynamic properties indicative of reperfusion injury.

In one embodiment, the measuring probe has an application face comprising a plurality of sensors for collecting a hemodynamic property of the blood vessel of the subject. In one embodiment, the application face is the side of the measuring probe contacting the subject. In one embodiment, the hemodynamic property collected by the measuring probe is the diameter of the blood vessel. In another embodiment, the hemodynamic property is blood flow velocity through the blood vessel being measured. In another embodiment, the hemodynamic property is flow mediated dilation (FMD), that is the change in blood vessel diameter incident to hyperemia such as by applying transient occlusion to the blood vessel. In another embodiment, the hemodynamic property is tissue pulsation around a blood vessel being measured.

In one embodiment, the plurality of sensors of the measuring probe are ultrasonic transducers. Depending on the embodiment, the ultrasonic transducers are imaging or non-imaging. In one embodiment, the ultrasonic transducers emit signals at frequencies between 5 to 10 megahertz. The higher frequency ranges of the ultrasonic transducers result in smaller wavelengths of the emitted signals. Embodiments placing the measuring probe proximate to the brachial artery are well suited for such frequency ranges, as the depth of the brachial artery and nature of the soft tissue surrounding the brachial artery permit the high frequency, low wavelength signals to still penetrate to the depth of the brachial artery without interference from surrounding tissue. Embodiments operating near 10 megahertz, or embodiments operating even higher, and the resulting smaller wavelengths permit more directional transmissions from the ultrasonic transducer and improve collection of the hemodynamic property as there is less noise to filter from other tissue interacting with the ultrasonic transducer signals.

To ensure a more directional signal transmission reaches a targeted blood vessel of a subject, embodiments of the invention serially align the ultrasonic transducers such that when applied to a subject, the serially aligned ultrasonic transducers are perpendicular to the target blood vessel. In other embodiments, the serially aligned ultrasonic transducers are staggered or placed in a circular array. The plurality of sensors ensure at least one sensor transmitting a directional signal reaches a target blood vessel.

In other embodiments, the plurality of sensors are pressure sensors. In one embodiment, the pressure sensors are constructed from polyvinylidene flouride. In other embodiments, the pressure sensors are polyvinylidene diflouride. Polyvinylidene flouride or polyvinylidene diflouride detect pulsation of the artery at the skin surface of the subject. Additionally, polyvinylidene flouride or polyvinylidene diflouride can listen to the flow of blood to determine blood flow velocity through the blood vessel being measured.

In another embodiment, the plurality of sensors use near infrared spectroscopy to determine the absorption by the blood vessel of near infrared light transmitted from the plurality of sensors. A calculation of the absorption and reflection of near infrared light uses the Doppler effect to determine the hemodynamic property of the underlying blood vessel. In another embodiment, the plurality of sensors are pulse oximeters transmitting light into the subject and measuring reflected light to determine distension of blood vessels as blood flows through the vessel.

In one embodiment, the measuring probe is coupled to a device for applying pressure to the blood vessel being measured. In one embodiment, the pressure applied occludes the flow of blood through the blood vessel. In one embodiment, the occlusion is transient. In one embodiment, the device applying pressure is a pneumatic cuff

In one embodiment, the measuring probe comprises reference indicia. The reference indicia in one embodiment is a visual cue for placing the measuring probe proximate to a blood vessel of a subject. In one embodiment, the visual cue is arrows or other symbols pointing to anatomical references such that when the anatomical reference (as an example of one embodiment aligning the measuring probe with a brachial artery, an anatomical reference is the armpit) aligns with the visual cue, the measuring probe is placed over a target blood vessel to be measured. In other embodiments, further reference indicia orient the measuring probe such that the alignment of the plurality of sensors are perpendicular to the orientation of the blood vessel. In other embodiments, the reference indicia is text explaining where to place the measuring probe or where to align the measuring probe's visual cues or markers otherwise on the measuring probe with anatomical or other external references.

In one embodiment, the device for applying pressure to the blood vessel being measured, such as a pneumatic cuff, comprises reference indicia. The reference indicia in one embodiment is a visual cue for placing the device for applying pressure proximate to a blood vessel that will be measured for a hemodynamic property. In one embodiment, the visual cue is arrows or other symbols pointing to anatomical references such that when the anatomical reference (for example, the crook of an elbow) aligns with the visual cue the device for applying pressure is placed at a consistent location independent of the particular subject applied to, or a particular user applying the device for applying pressure. In other embodiments, the reference indicia is text explaining where to place the device for applying pressure or where to align the visual cues or markers otherwise.

In one embodiment, the device for applying pressure has a mounting structure for receiving and housing a measuring probe. In such an embodiment, the measuring probe is placed into the mount, and has visual cues on the measuring probe aligning placement in the mount to orient the plurality of sensors perpendicular to a target blood vessel. In these embodiments, the mount is an “external reference” that the measuring probe aligns with. In one embodiment, the mount is internal to the edges of the device for applying pressure such that the measuring probe, when in the mount, is embedded within the device for applying pressure. In other embodiments, the mount is external to the edges of the device for applying pressure such that the measuring probe is distal to the device for applying pressure when coupled to the mount.

In another embodiment, the measuring probe is physically connected to a device for applying pressure, such as a pneumatic cuff, such as by the mount discussed previously, and in another embodiment is electronically coupled to the device for applying pressure. In one embodiment, the electronic coupling is through a reperfusion injury analysis device. In one embodiment, the reperfusion injury analysis device comprises a controller. In one embodiment, the controller regulates the amount and duration of pressure delivered to the blood vessel by the device for applying pressure. In one embodiment, the controller dictates the application of pressure relative to measuring a hemodynamic property of a blood vessel by the measuring probe. In one embodiment, the controller dictates application and release of pressure to produce transient occlusion to the blood vessel.

In one embodiment, the transient occlusion is subsequent to an initial measurement of the hemodynamic property. In another embodiment, the transient occlusion is prior to a measurement of the hemodynamic property. In still another embodiment, the measuring probe begins collecting hemodynamic properties of the blood vessel, the controller applies transient occlusion to the blood vessel through the device for applying pressure, and after occlusion the measuring probe continues to measure the hemodynamic property.

In another embodiment, the application face of the measuring probe includes an offset apparatus. In one embodiment, the offset apparatus is an angled wedge on the application face with an embedded plurality of sensors. In embodiments with an offset apparatus, the plurality of sensors, such as ultrasonic transducers, deliver signals into the subject such that the signals are not delivered perpendicular to a flow of blood in the blood vessel but instead orient against a linear flow of blood in the blood vessel. In these embodiments, Doppler effect principles are enhanced for measuring hemodynamic properties. In one embodiment, the offset apparatus is a reference indicia itself, such that when the application face of the measuring probe is applied to a subject, the plurality of sensors transmit signals against the flow of blood. For exemplary purposes only, for an embodiment measuring flow of blood in a brachial artery, the offset apparatus angles the application face towards the armpit, and can include a visual cue like an arrow pointing towards the armpit of the subject, such that signals emitted from the plurality of sensors transmit towards the heart while the flow of blood is away from the heart. Such embodiments help discern flow of blood through an artery from the flow of blood through a vein.

In another embodiment, the reperfusion injury analysis device further comprises a data module. The data module, in one embodiment, receives a collected hemodynamic property from the measuring probe and performs measurements. In one embodiment, the measurement is a vessel diameter determined by the Doppler effect as signals from the measuring probe are transmitted into and reflected by the blood vessel of the subject. In another embodiment, the measurement is a flow of blood in the blood vessel. In still another embodiment, the measurement is a visual depiction of the blood vessel or the blood flow velocity. The different measurements enabled by the various embodiments are a function of the different frequencies received by the plurality of sensors that indicate different matter interacting with the signals transmitted by the measuring probe.

In another embodiment, the data module calculates a metric of the hemodynamic property. In one embodiment, the calculated metric is a difference of the hemodynamic property before transient occlusion is applied and the hemodynamic property after transient occlusion is applied; in another embodiment the calculated metric is the amount of time the post-occluded hemodynamic property takes to return to the pre-occluded hemodynamic property levels. In one embodiment, the calculated metric is FMD as determined by comparative calculation of the changes in diameter of the blood vessel being measured before, during, or after transient occlusion. In still other embodiments, the calculated metric is FMD as a function of time in the presence of transient occlusion.

In another embodiment, the reperfusion injury analysis device comprises an output module. In embodiments with imaging ultrasonic transducers, the output device is a graphic user interface displaying a simulated view of blood vessel and/or the hemodynamic property over time. In another embodiment, the output module displays a textual representation of a calculated metric as calculated by the data module of the reperfusion injury analysis device. Such calculated metrics include, in one embodiment, numerical ranges of the hemodynamic property of the blood vessel in pre- and post-occluded states.

In one embodiment, a hydrogel adhesive facilitates application of the measuring probe to the subject and fixes the measuring probe in place during its collection of a hemodynamic property. Embodiments with adhesives preclude a user, such a caregiver, from continuously holding the measuring probe in place while the hemodynamic property is collected by the measuring probe. In another embodiment, the hydrogel adhesive enhances transmission of signals from, and reflection of signals to, the measuring probe by providing a contact interface with the subject that has improved signal impedance properties relative to air for transmitting signals through the multiple mediums between the measuring probe and the blood vessel being measured. The hydrogel adhesive further provides a constant surface on the subject by reducing air pockets or uneven surfaces on the subject such as through dry skin, thereby reducing static.

In one embodiment, the hydrogel adhesive comprises medical grade hydrogel. In one embodiment, the hydrogel is arranged on the interface element in variable layers and orientations to angle signals to or from the measuring probe in parallel, or near parallel, with a blood vessel of a subject.

Another embodiment of the invention is a method for analyzing reperfusion injury in a subject. In one embodiment, the method begins by applying a measuring probe to a blood vessel, such as a major artery like a brachial artery, of a subject that has experienced, or is experiencing, a cardiac distress event. In one embodiment, the measuring probe is affixed to the upper arm of the subject to measure a hemodynamic property of a brachial artery.

In one embodiment, a hemodynamic property of the blood vessel is measured. In one embodiment, measurement is by ultrasonic transducers. Other embodiments may measure the hemodynamic property by other means, such as those described elsewhere in this disclosure, and one having skill in the art will envision further hemodynamic property measurement means. In another embodiment, a device for applying pressure to a blood vessel being measured is applied to the subject. In one embodiment, the device for applying pressure in a pneumatic cuff. In one embodiment, the pressure applied to the blood vessel being measured is temporary and produces transient occlusion. In some embodiments, the measurement of the hemodynamic property of the blood vessel before the transient occlusion is applied (a pre-occluded hemodynamic property) is stored in a data module of a reperfusion injury analysis device coupled to the measuring probe. In another embodiment, after a pneumatic cuff releases transient occlusion to the blood vessel, a post-occluded hemodynamic property of the blood vessel is measured.

In one embodiment, the pre-occluded and post-occluded hemodynamic property of the blood vessel is analyzed by a reperfusion injury analysis device and a metric is calculated. In one embodiment, the calculated metric is a difference of the hemodynamic property from the post-occluded state to the pre-occluded state. For example, in one embodiment, the metric is FMD (again, the change in diameter of the blood vessel in an occluded or post-occluded state relative to a pre-occluded state). In another example, the metric is the percent change in blood flow velocity of blood through the blood vessel in a post-occluded state relative to a pre-occluded state. In still another embodiment, the calculated metric is the time required for the post-occluded hemodynamic property to return to a pre-occluded status.

In another embodiment, the calculated metric is communicated to an external source. In one embodiment, the external source is an output module of a reperfusion injury analysis device. In one embodiment, the output module is a graphic user interface displaying images of the hemodynamic property such as an image of the blood vessel. In one embodiment, the graphic user interface displays the hemodynamic property as a function of time. For example, in one embodiment the diameter of the blood vessel is displayed, as calculated by the reperfusion injury analysis device, from a pre-occluded state through to a post-occluded state. In another embodiment, the calculated metric is displayed as a textual representation of the hemodynamic property measured, and in another embodiment includes a numerical difference between the pre-and post-occluded states.

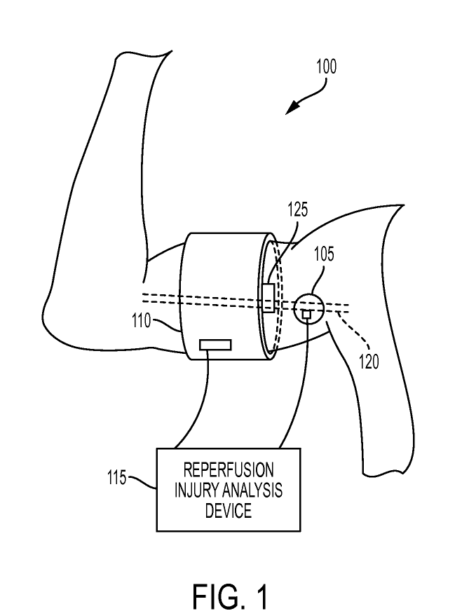

Turning now to the figures, FIG. 1 illustrates an embodiment of reperfusion device 100. Reperfusion device 100 includes measuring probe 105, and as depicted measuring probe 105 is place over brachial artery 120, though one of skill in the art will envision other blood vessels to apply measuring probe 105 to. Reperfusion device 100 further includes pneumatic cuff 110 in the depicted embodiment, though other devices for applying pressure may be suitable. Pneumatic cuff 110 further includes, in one embodiment, mount 125. Mount 125 is configured to house measuring probe 105 distal to pneumatic cuff 110 according to one embodiment.

Reperfusion device 100 further includes reperfusion injury analysis device 115. Measuring probe 105 is coupled, in one embodiment, to reperfusion injury analysis device 115 electronically either by wired connection or by wireless connection such as near field communication or network connection. Pneumatic cuff 110 is coupled, in one embodiment, to reperfusion injury analysis device 115 electronically either by wired connection or by wireless connection such as near field communication or network connection. In one embodiment, measuring probe 105 is operably coupled to pneumatic cuff 110 by direct wired connection or by wireless connection such as near field communication or network connection.

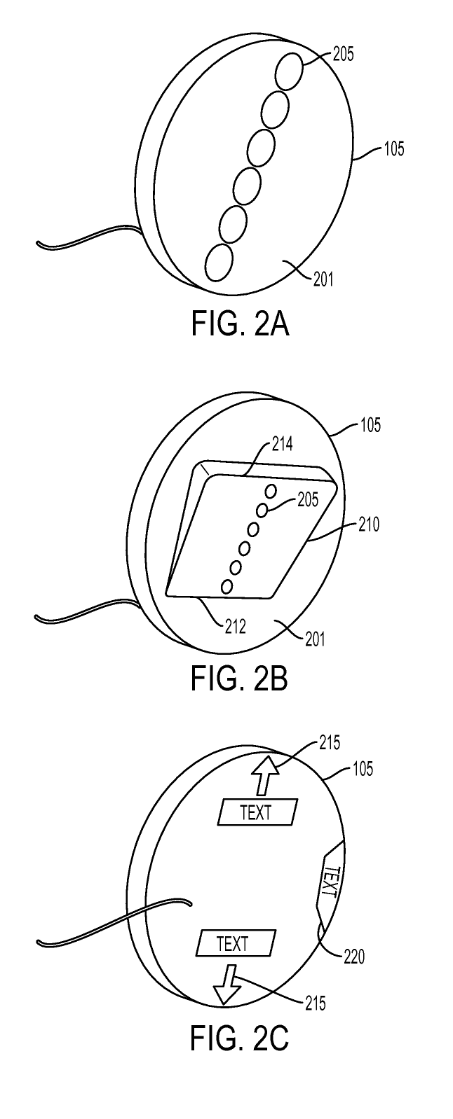

FIG. 2A illustrates one embodiment of application face 201 of measuring probe 105. In one embodiment, application face 201 is embedded with a plurality of sensors 205. In one embodiment, plurality of sensors 205 are ultrasonic transducers. As depicted, in one embodiment, plurality of sensors 205 are linearly aligned; in another embodiment, plurality of sensors 205 is aligned in a staggered linear array or arranged in a circular array. One of skill in the art can envision alternative layouts of plurality of sensors 205.

FIG. 2B illustrates another embodiment of application face 201 of measuring probe 105, with offset apparatus 210 housing plurality of sensors 205. In one embodiment, as shown in FIG. 2B, offset apparatus 210 is a wedge shape to angle, when applied, application face 201 relative to the surface of a subject, thereby directing the transmission of signals from plurality of sensors 205 against a flow of blood in a blood vessel of a subject.

In one embodiment, offset apparatus 210 is a wedge with a lower end 212 and a higher end 214. In one embodiment, lower end 212 is placed further away from the heart of a subject relative to higher end 214; in such embodiments this placement directs the transmission of signals from plurality of sensors 205 more towards the heart of a subject and against the flow of arterial blood away from the heart. In one embodiment, lower end 212 and higher end 214 are reference indicia to measuring probe 105 by guiding orientation of applying measuring probe 105 to a subject.

FIG. 2C illustrates an embodiment of measuring probe 105 with reference indicia. In one embodiment, reference indicia are visual cues 215 to align with or point to anatomical or external references. In one embodiment, visual cues 215 point to an anatomical reference such as an armpit of a subject for embodiments that place measuring probe 105 over a brachial artery 120 (not shown, but depicted in FIG. 1 as brachial artery 120). Visual cues 215 in another embodiment are arrows pointing to the bicep muscle and/or the triceps muscles to guide application of measuring probe 105. In another embodiment, visual cues 215 guide measuring probe to a mount (not shown, but depicted in FIG. 1 as mount 125). In still other embodiments, reference indicia are text 220 explaining where to place measuring probe 105.

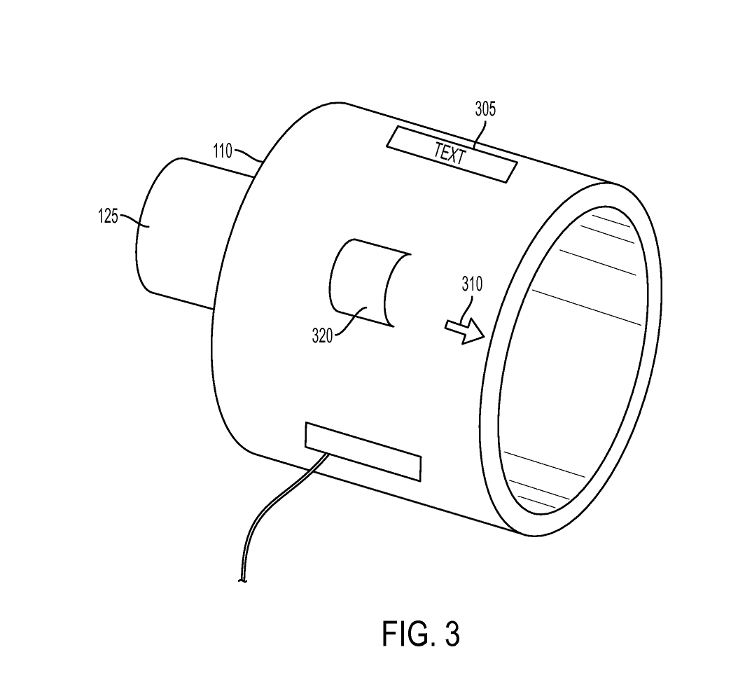

FIG. 3 illustrates an embodiment of a device for applying transient occlusion to a blood vessel of a subject. In one embodiment, the device for applying transient occlusion to a blood vessel is pneumatic cuff 110 as similarly depicted in FIG. 1. In one embodiment, pneumatic cuff 110 has reference indicia to guide placement of pneumatic cuff 110 on a subject. In one embodiment, reference indicia is visual cue 310 aligning a point on pneumatic cuff 110 to an anatomical reference, such as the crook of an elbow. In another embodiment, reference indicia is text 305 explaining where to place pneumatic cuff 110. In one embodiment, pneumatic cuff 110 includes mount 125 for coupling with a measuring probe and serving as an external reference for guiding placement of measuring probe 105. In one embodiment, mount 125 is distal to the interior of pneumatic cuff 110, but in other embodiments mount 125 is located within the edges of pneumatic cuff 110 at internal mount point 320. Internal mount point 320 may be a sleeve to slide measuring probe 105 into, or a cutout of the material of pneumatic cuff 110 to place measuring probe 105 into.

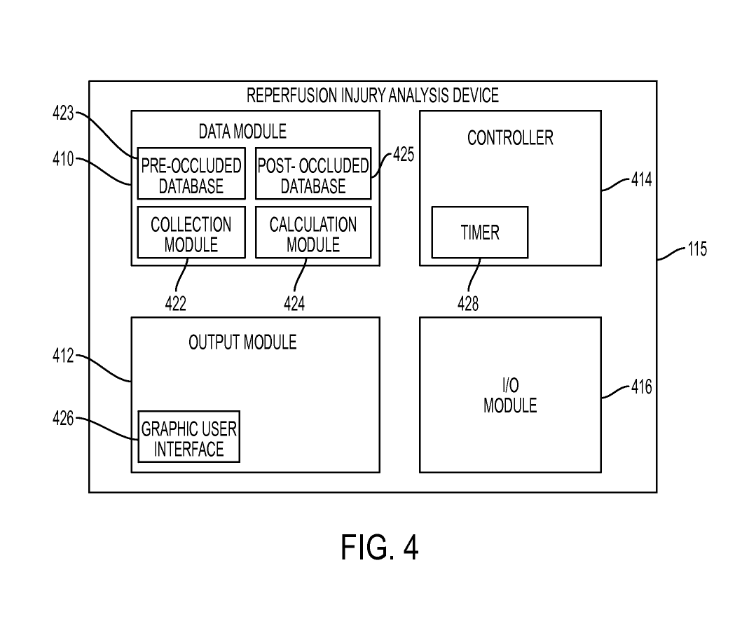

FIG. 4. illustrates a system diagram of an embodiment of a reperfusion injury analysis device 115. In one embodiment, reperfusion injury analysis device 115 comprises data module 410. In one embodiment, reperfusion injury analysis device 115 is operably coupled to measuring probe 105 (not shown) and receives hemodynamic properties collected by measuring probe 105. Coupling to measuring probe 105 in one embodiment is through wired connection, in alternative embodiments is through wireless connection such as network connection or near field communication. Data module 410 receives the hemodynamic properties in a collection module 422. The collection module 422 in one embodiment categorizes the collected property relative to transient occlusion of the blood vessel being measured; in one embodiment, collected hemodynamic properties before transient occlusion are stored in a pre-occluded database 423 whereas in alternative embodiments, a post-occluded database 425 stores hemodynamic properties collected after transient occlusion.

In one embodiment, a calculation module 424 analyzes the pre-occluded and post-occluded hemodynamic properties. In one embodiment, calculation module 424 calculates a metric between the pre-occluded and post-occluded hemodynamic properties. In one embodiment, the calculated metric determined by calculation module 424 is a percentage change in diameter of the blood vessel being measured. In another embodiment, the calculated metric determined by calculation module 424 is a percentage change in blood flow velocity through the blood vessel being measured. In still other embodiments, the calculated metric determined by calculation module 424 is the time for a post-occluded hemodynamic property to return to the measured pre-occluded state.

For example purposes only of the interaction between collection module 422 and calculation module 424, in one embodiment signals received by a transducer transmitting to a brachial artery are collected from measuring probe 105 and received by data module 410 in collection module 422. Transient occlusion is applied to the brachial artery and after the occlusion is released, measuring probe 105 transmits and collects the post-occluded signals of the brachial artery. Data module 422 receives the post-occluded signals in collection module 422, and then delivers both the pre-occluded and post-occluded signals to calculation module 424. Calculation module 424 analyzes the data, and in one embodiment interprets the signals to determine diameter of the brachial artery in pre- and post-occluded states. Calculation module 424 then determines a percentage change between the pre- and post-occluded states and calculates a metric of FMD. In other embodiments, instead of calculating a diameter from the received signals, calculation module 424 determines a blood flow velocity from the signals received in collection module 422 at the various times relative to occlusion, or a percentage change in the blood flow velocity, or the time for a post-occluded hemodynamic property to return to a pre-occluded state.

In one embodiment, reperfusion injury analysis device 115 comprises an output module 412. Output module 412 is configured to communicate the calculated metric as determined by calculation module 424. In one embodiment, output module 412 includes a graphic user interface 426. In one embodiment, output module 412 displays the calculated metric of calculation module 424 on graphic user interface 426 as a real time image of the blood vessel being measured. In one embodiment, output module 412 displays the calculated metric of calculation module 424 on graphic user interface 426 as a numerical representation of the calculated metric, such as the percentage change of blood vessel diameter or percentage change of blood flow velocity relative to pre- and post-occluded collections by measuring probe 105.

In another embodiment, reperfusion injury analysis device 115 comprises a controller 414 to provide consistent pressure and duration of transient occlusion applied from a device to apply pressure (not shown), such as pneumatic cuff 110 as depicted in FIG. 1. Controller 414 dictates when pneumatic cuff 110 inflates to apply transient occlusion and how long the occlusion lasts to reduce user variability in determining a proxy metric of reperfusion injury. In one embodiment, the timing of inflation and duration is dictated by timer 428. In one embodiment, timer 428 is programmed such that when the reperfusion injury analysis device 115 is activated by a user at I/O module 416, the timer provides a pre-occlusion measurement period to collect the hemodynamic property of a blood vessel, then initiates an occlusion start time to controller 414 to apply pressure to the blood vessel through a pneumatic cuff 110, then initiates a post-occlusion time to remove pressure from pneumatic cuff 110 and begin a post-occlusion measurement period to collect the hemodynamic property of the blood vessel in a post-occluded state.

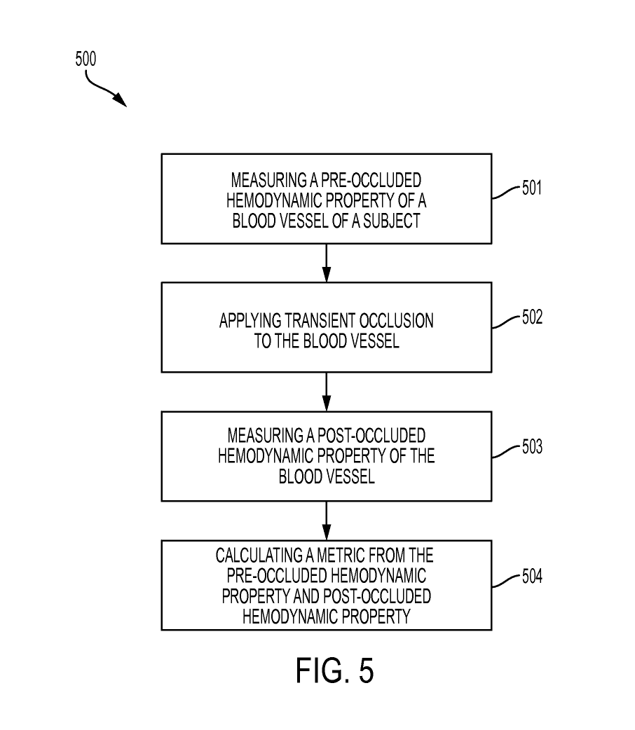

FIG. 5 illustrates an exemplary embodiment of a method 500 for analyzing reperfusion injury in a subject that has experienced, or is experiencing, a cardiac distress event. In one embodiment, method 500 begins by measuring a hemodynamic property of a blood vessel of a subject at 501. The hemodynamic property may be blood vessel diameter, or blood flow velocity, or tissue pulsation according to various embodiments. One of skill in the art will envision alternative hemodynamic properties suitable for measurement in context of this disclosure. In one embodiment, transient occlusion is applied to the blood vessel at 502. After transient occlusion is applied, the hemodynamic property is measured again at 503. At 504, a metric from the pre- and post-occluded hemodynamic properties is calculated. In one embodiment, the calculated metric is FMD; in another embodiment the calculated metric is the percentage change in blood flow velocity. In still other embodiments, calculating the metric is determining the amount of time required to return the post-occluded hemodynamic property to the pre-occluded hemodynamic property state. The particular calculated metric varies across embodiment as the measured hemodynamic property varies with embodiment.

It should be appreciated that the specific steps illustrated in FIG. 5 provide a particular sequence of calculating a metric of reperfusion injury. Other sequences of steps may also be performed according to alternative embodiments. For example, alternative embodiments of the present invention may perform the steps outlined above in a different order. Moreover, the individual steps illustrated in FIG. 5 may include multiple sub-steps as appropriate to the individual step. For example, in one embodiment measuring the hemodynamic property may also occur during application of transient occlusion at step 502, such that measurement of the blood vessel's hemodynamic property is continuous throughout method 500. Furthermore, additional steps may be added, or certain steps may be removed, depending on the particular embodiments. One of skill in the art would recognize many variations, modifications, and alternatives.

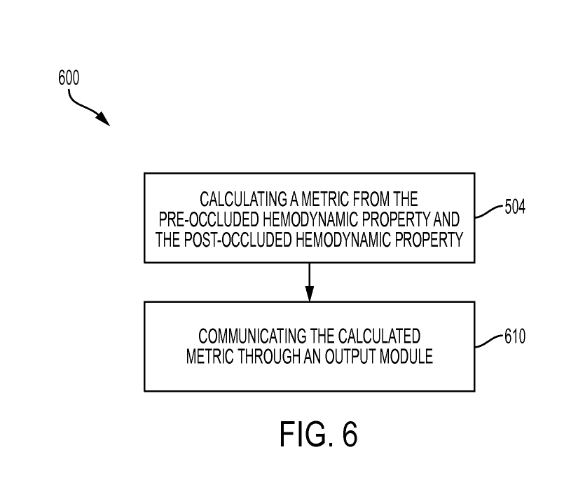

FIG. 6 illustrates an exemplary process of a method 600. Method 600 is a continuation of method 500, beginning at 504 by calculating a proxy metric of reperfusion injury as determined from pre-occluded and post-occluded states of a hemodynamic property in a subject. In method 600, step 504 is followed by step 610 in which the calculated metric is communicated on an output module. According to embodiment, communicating the metric may be as a real time image of the hemodynamic property on a graphic user interface, such as an image of the blood vessel being measured or a graph of the hemodynamic property over time; in other embodiments the communication is by numerical display of the calculated metric.

While the invention has been described in terms of particular embodiments and illustrative figures, those of ordinary skill in the art will recognize that the invention is not limited to the embodiments or figures described. For example, in various embodiments described above, a reperfusion injury analysis device receives collected hemodynamic properties in blood vessels and calculates metrics relative to transient occlusion of those blood vessels to communicate the calculated metric on an output module of the reperfusion injury analysis device. However, in other embodiments, the reperfusion injury analysis device communicates the calculated metric to a third party, such as a medical facility or primary care giver to provide follow on care information regarding the status and extent of reperfusion injury in the subject. In still other embodiments, the calculated metric is stored on a third party system and subsequent collection of calculated metrics across a plurality of subjects is associated with survival rates of those subjects' experiences with cardiac distress events such that the data module can access the third party system to determine whether the calculated metric on an instant subject places the subject in a particular risk category.

Reference throughout this document to “one embodiment,” “certain embodiments,” “an embodiment,” or similar term means that a particular feature, structure, or characteristic described in connection with the embodiment is included in at least one embodiment. Thus, the appearances of such phrases in various places throughout this specification are not necessarily all referring to the same embodiment. Furthermore, the particular features, structures, or characteristics may be combined in any suitable manner on one or more embodiments without limitation.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one,” “at least one” or “one or more.” Unless otherwise required by context, singular terms used herein shall include pluralities and plural terms shall include the singular.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” The term “or” as used herein is to be interpreted as inclusive or meaning any one or any combination. Therefore, “A, B or C” means any of the following: A; B; C; A and B; A and C; B and C; A, B and C. An exception to this definition will occur only when a combination of elements, functions, steps or acts are in some way inherently mutually exclusive.

Words using the singular or plural number also include the plural and singular number, respectively. Additionally, the words “herein,” “above,” and “below” and words of similar import, when used in this disclosure, shall refer to this disclosure as a whole and not to any particular portions of the disclosure.

The description of embodiments of the disclosure is not intended to be exhaustive or to limit the disclosure to the precise form disclosed. While specific embodiments and examples for the disclosure are described herein for illustrative purposes, various equivalent modifications are possible within the scope of the disclosure, as those skilled in the relevant art will recognize. Such modifications may include, but are not limited to, changes in the dimensions and/or the materials shown in the disclosed embodiments.

All of the references cited herein are incorporated by reference. Aspects of the disclosure can be modified, if necessary, to employ the systems, functions, and concepts of the above references to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Therefore, it should be understood that the invention can be practiced with modification and alteration within the spirit and scope of the appended claims. The description is not intended to be exhaustive or to limit the invention to the precise form disclosed. It should be understood that the invention can be practiced with modification and alteration and that the invention be limited only by the claims and the equivalents thereof

What is claimed is:

1. A reperfusion device comprising:
a measuring probe having an application face and comprising a plurality of sensors configured to collect a hemodynamic property of a blood vessel of a subject contacting the measuring probe;
a pneumatic cuff coupled to the measuring probe configured to apply transient occlusion to the blood vessel being measuring;
a reperfusion injury analysis device operably coupled to the measuring probe and pneumatic cuff, the reperfusion injury analysis device comprising:
a data module configured to measure the collected hemodynamic property in the blood vessel being measured and calculate a proxy metric of reperfusion injury; and
an output module configured to communicate the calculated metric.

2. The reperfusion device of claim 1, wherein the plurality of sensors comprise a plurality of ultrasonic transducers.

3. The reperfusion device of claim 2, wherein the plurality of ultrasonic transducers emit a frequency between about five and about ten megahertz.

4. The reperfusion device of claim 1, further comprising a controller coupled to the pneumatic cuff, the controller programmed to apply transient occlusion to the blood vessel being measured.

5. The reperfusion device of claim 3, wherein the application face of the measuring probe further comprises an offset apparatus to position the application face at an angle relative to a surface on the subject contacting the measuring probe.

6. The reperfusion detection device of claim 5, wherein the offset apparatus positions the application face towards a direction opposite the direction of a flow of blood in the blood vessel of the subject being measured.

7. The reperfusion detection device of claim 6, further comprising a first reference indicia coupled to the measuring probe.

8. The reperfusion device of claim 7, wherein the first reference indicia is a visual cue which when aligned with an external reference positions the measuring probe to measure the flow of blood in the blood vessel being measured.

9. The reperfusion device of claim 6, further comprising a second reference indicia coupled to the pneumatic cuff.

10. The reperfusion detection device of claim 9, wherein the second reference indicia is a visual cue which when aligned with an external reference positions the pneumatic cuff to an occlusion position relative to the measuring probe.

11. The reperfusion device of claim 1, further comprising an adhesive hydrogel for fixing the measuring probe to the subject and propagating signals from the measuring probe to the blood vessel of the subject.

12. The reperfusion device of claim 1, wherein the hemodynamic property measured by the plurality sensors is flow mediated dilation as a function of time.

13. The reperfusion device of claim 1, wherein the blood vessel in a subject is a brachial artery.

14. The reperfusion device of claim 1, wherein the output module is a graphic user interface configured to display the measured hemodynamic property.

15. A method of analyzing reperfusion injury, the method comprising:
measuring a pre-occluded hemodynamic property of a blood vessel of a subject;
applying transient occlusion to the blood vessel being measured;
measuring a post-occluded hemodynamic property of the blood vessel being measured; and
calculating a metric from the pre-occluded hemodynamic property and the post-occluded hemodynamic property.

16. The method of claim 15, further comprising communicating the calculated metric on an output module.

17. The method of claim 16, wherein the output module is a graphic user interface.

18. The method of claim 17, wherein the calculated metric displayed is flow mediated dilation as a function of time.

19. The method of claim 15, wherein measuring the hemodynamic property is by ultrasonic transducers.

20. The method of claim 15, wherein calculating the metric is determining the time for the post-occluded hemodynamic property measurement to return to the pre-occluded hemodynamic property measurement status.
